(19) United States
(12) Patent Application Publication
(10) Pub. No.: US 2014/0053840 A1
(43) Pub. Date: Feb. 27, 2014

(54) Human-Machine Synchronization Method And Device Of Invasive Ventilator Operating In Noninvasive Ventilation Mode

(71) Applicant: BEIJING AEONMED CO., LTD., Beijing (CN)

(72) Inventor: Qingping Liu, Beijing (CN)

(21) Appl. No.: 14/005,651

(22) PCT Filed: Dec. 25, 2012
(86) PCT No.: PCT/CN2012/087395

§ 371 Date: Nov. 14, 2013

(30) Foreign Application Priority Data
Dec. 30, 2011 (CN) 201110455881.2

(51) Int. Cl. A61M 16/00; A61B 5/00; A61B 5/087

(52) U.S. Cl. CPC A61M 16/0051; A61M 16/0057; A61B 5/087; A61B 5/7225; A61B 5/4836
USPC 128/204.23

(57) ABSTRACT

A human-machine synchronization method and device of an invasive ventilator operating in a noninvasive ventilation mode. The method includes steps of: measuring an airway pressure, an inspiratory flow, and an expiratory flow; calculating a gas leakage flow according to a pre-established gas leakage estimation model and by using the airway pressure, the inspiratory flow and the expiratory flow; and compensating a basic flow according to the gas leakage flow. In the above method, the gas leakage flow is estimated by means of the gas leakage estimation model, to compensate the gas leakage, thereby facilitating the noninvasive ventilation of the invasive ventilator and improving the human-machine synchronization.

### FIELD OF THE INVENTION

The invention relates to the technical field of a ventilator, in particular to a human-machine synchronization method and device of an invasive ventilator operating in a non-invasive ventilation mode.

### BACKGROUND OF THE INVENTION

Ventilators include invasive ventilators and non-invasive ventilators according to their modes of connections with patients, the invasive ventilator is connected to the patient by an airway intubation, and the non-invasive ventilator is connected to the patients by a face mask or a nasal mask. The invasive ventilator, such as a traditional Intensive Care Unit (ICU) ventilator, generally adopts a double-limb ventilation circuit with an expiratory valve, which was initially designed for invasive ventilation. With the widely use of the non-invasive ventilation (NIV), a non-invasive ventilation mode is introduced to the invasive ventilator. The NIV mode refers to a mode in which patients with an autonomous breathe capability are ventilated by a human-machine interface, such as the face mask or the nasal mask, that is different from the airway intubation. The general NIV mode includes any mode suitable for invasive ventilation, such as a Pressure Support Ventilation (PSV) mode, a Pressure Control Ventilation (PCV) mode and a Volume Control Ventilation (VCV) mode, but in a narrow sense, the NIV model refers to the PSV mode.

Generally, a patients experiencing the non-invasive ventilation is conscious and can autonomously breathe, thus it is demanding for the synchronization of a human-machine interaction during the non-invasive ventilation, that is, it is required that the beginning of the inspiration (triggering) and the beginning of the expiration (switching) is controlled by the patient. The ideal human-machine synchronization is that the patient supported by the ventilator can freely breathe in an unimpeded state without the control of the ventilator, just like normal people. The comfort and tolerance of the patient can be improved by the human-machine synchronization, thereby ensuring the success of the ventilation treatment. Clinical studies showed that there is a high failure rate of the non-invasive ventilation, and one of the important factors for this is the human-machine asynchrony.

The main reason causing the human-machine asynchrony is an inevitable gas leakage at the human-machine interface. Because the traditional trigger (a conversion from the expiration to the inspiration) judgment technology based on a basic flow and the traditional switching (a conversion from the inspiration to the expiration) judgment technology based on a peak flow percentage depend on an accurate measurement of a pulmonary flow, the presence of the gas leakage causes an incorrect estimation on the pulmonary flow, thereby causing an asynchronous trigger and asynchronous switching.

The non-invasive ventilator generally, which adopts a structure with a single-limb ventilation circuit without an expiratory valve and uses special gas leakage compensation technologies and trigger/switching judgment technologies, shows relative good human-machine synchrony. However, due to the differences in aspects such as structure, control algorithm, and trigger/switching mechanism between the dedicated non-invasive ventilator and the traditional invasive ventilator, the human-machine synchronization technology adopted by the non-invasive ventilator cannot be seamlessly transferred to the traditional invasive ventilator. Therefore, it is a technical problem to design an effective human-machine interaction mechanism to improve the synchrony of the traditional invasive ventilator operating in the NIV mode.

### SUMMARY OF THE INVENTION

An object of the present invention is to improve the human-machine synchrony of a traditional invasive ventilator operating in the NIV mode.

In order to achieve the above object, the present invention provides the following technical solution.

A human-machine synchronization method of an invasive ventilator operating in a non-invasive ventilation mode, comprising: measuring an airway pressure, an inspiratory flow and an expiratory flow; calculating a gas leakage flow based on the airway pressure, the inspiratory flow and the expiratory flow according to a pre-established gas leakage estimation model; and compensating a basic flow according to the gas leakage flow.

Preferably, the method above further comprising: recording switching time and trigger time of the current respiration at the end of expiration, and iteratively learning a trigger/switching cycle by means of digital filtering when the switching time and the trigger time of the current respiration respectively differentiate from the switching time and trigger time of the preceding respiration by less than a preset difference, to obtain an autonomy trigger/switching cycle of a patient.

Preferably, the method above further comprising: setting the trigger/switching threshold to be a threshold with a high sensitivity at the autonomy trigger/switching time point and to be a threshold with a low sensitivity at other time points according to the autonomous trigger/switching cycle of the patient.

Wherein updating parameters of the gas leakage estimation model according to the latest autonomy trigger/switching cycle of the patient.

Preferably, the method above further comprising: judging, at the end of the inspiration, whether gas leakage is exceptionally increased according to the current inspiratory tidal volume and the preceding inspiratory tidal volume, and if so, modifying the basic flow and the trigger threshold of the expiratory phase.

Wherein the criteria of trigger judgment comprise variations of the expiratory flow gradient or an expiratory filtering pressure with different time constants for pressure filtering; and criteria of switching judgment comprise an inspiratory filtering pressure with different time constants for pressure filtering.

Wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model; kₗ is calculated by the following parameter estimation model of

$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$

wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations.

Wherein, N takes a larger value when the gas leakage is stable and takes a smaller value when the gas leakage is increased/decreased exceptionally.

A device for human-machine synchronization of an invasive ventilator operating in a non-invasive ventilation mode, comprising: a measuring unit, which is used for measuring an airway pressure, an inspiratory flow and a respiratory flow; a gas leakage estimation unit, which is used for calculating a gas leakage flow based on the airway pressure, the inspiratory flow and the expiratory flow measured by the measuring unit according to a pre-established gas leakage estimation model; and a compensation unit, which is used for compensating a basic flow according to the gas leakage flow.

Preferably, the device above further comprising: an autonomous trigger/switching cycle learning unit, which is used for recording switching time and trigger time of the current respiration at the end of expiration, iteratively learning a trigger/switching cycle by means of digital filtering when the switching time and the trigger time of the current respiration respectively differentiate from the switching time and the trigger time of the preceding respiration by less than a preset difference to obtain an autonomy trigger/switching cycle of a patient, and updating parameters of the gas leakage estimation model according to the latest autonomy trigger/switching cycle of the patient.

Preferably, the device above further comprising: a trigger/switching threshold updating unit, which is used for setting the trigger/switching threshold to be a threshold with a high sensitivity at the autonomy trigger/switching time point and to be a threshold with a low sensitivity at other time points according to the autonomous trigger/switching cycle of the patient obtained by the autonomous trigger/switching cycle learning unit Preferably, the device above further comprising: a gas leakage exception handling unit, which is used for judging, at the end of the inspiration, whether gas leakage is exceptionally increased according to the current inspiratory tidal volume and the preceding inspiratory tidal volume, and if so, instructing the compensation unit and the trigger/switching threshold updating unit to adjust the basic flow and the trigger threshold of the expiratory phase.

Wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model; kₗ is calculated by the following parameter estimation model of

$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$

wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations.

As can be seen in the present invention, the gas leakage flow is estimated by the gas leakage estimation model to compensate the gas leakage, for the purposes of improving the non-invasive ventilation of the invasive ventilator and improving the human-machine synchrony.

The parameters of the gas leakage estimation model are updated in each respiration cycle, and the number of respirations needed by the parameter estimation is adaptively selected according to the gas leakage variation, thereby achieving the relatively accurate gas leakage estimation by using the estimated parameters. Further, the basic flow and the trigger threshold are adjusted when the gas leakage is exceptionally increased, thereby assuring relatively accurate estimation of the gas leakage flow and the pulmonary flow even in the case of a large gas leakage or a gas leakage exception.

Further, the present invention provides the mechanism of learning the autonomous trigger/switching cycle of the patient, so that the autonomous trigger/switching cycle of the patient can be used as a basis of the trigger/switching threshold. The autonomous trigger/switching threshold is not fixed, but varied with time, thereby reducing the trigger/switching power of the patient maximally while preventing an incorrect trigger/switching.

In addition, the variation of the expiratory flow gradient is used as the criteria of the trigger judgment in the present invention, to avoid the affection of the gas leakage on the trigger, thus further improving the performance of the man-machine synchrony. Meanwhile, the difference between the PEEP acquired by the filtering algorithm and the actual airway pressure is taken as another criterion of the trigger judgment, to avoid the affection of the gas leakage on the trigger, thus improving the human-machine trigger synchrony.

### BRIEF DESCRIPTION OF THE DRAWINGS

FIG. 1 is a flow chart of a human-machine synchronization method of an invasive ventilator operating in a non-invasive ventilation mode according to the present invention;

FIG. 2 is a schematic diagram showing the operating of the invasive ventilator in the non-invasive ventilation mode according to the present invention;

FIG. 3 is a flow chart of a method according to an embodiment of the present invention;

FIG. 4 is a flow chart of autonomous respiration cycle learning of the method according to the embodiment of the present invention;

FIG. 5 is a schematic diagram showing the variation of an autonomous trigger/switching threshold in the method according to the embodiment of the present invention;

FIG. 6 is a schematic diagram showing a variation of the expiratory flow gradient in the method according to the embodiment of the present invention;

FIGS. 7a and 7b are schematic diagrams showing the filtering of the expiratory phase pressure in the method according to the embodiment of the present invention; and

FIG. 8 is a schematic diagram showing the structure of the device corresponding to the method according to the embodiment of present invention.

### DETAILED DESCRIPTION OF THE EMBODIMENTS

The present invention provides a human-machine synchronization solution for an invasive ventilator (such as a traditional ICU ventilator) operating in a NIV mode, which specifically estimates a gas leakage at the human-machine interface by a pre-established gas leakage estimation model, and then compensates a basic flow according to the gas leakage flow, thus improving the human-machine synchrony.

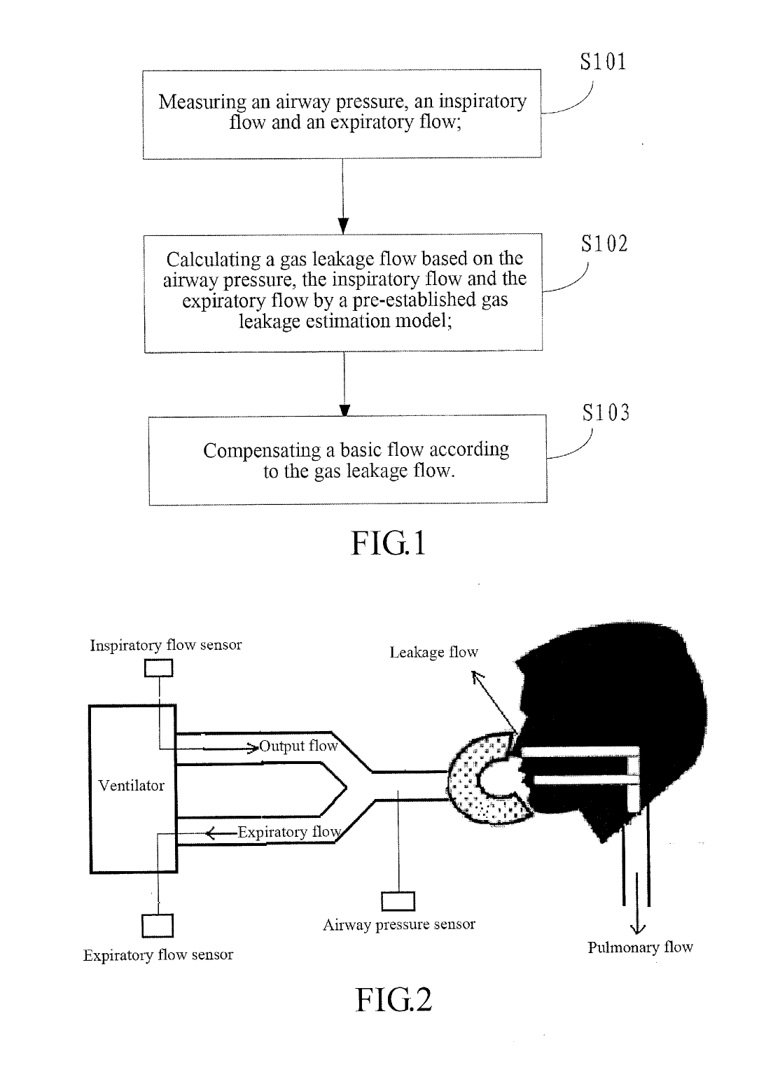

Referring to FIG. 1, which shows a flow chart of the human-machine synchronization method of an invasive ventilator operating in the non-invasive ventilation mode according to the present invention, and the method includes the following steps:

S101: measuring an airway pressure, an inspiratory flow and an expiratory flow;

S102: calculating a gas leakage flow according to the airway pressure, the inspiratory flow and the expiratory flow based on a pre-established gas leakage estimation model; and

3103: compensating a basic flow according to the gas leakage flow.

Further, the present invention improves the judgment on synchronous trigger/switching, including such a design of an autonomous flow trigger/switching threshold based on an autonomous respiration cycle learning mechanism of a patient, an inspiratory trigger judgment based on a variation of the expiratory flow gradient, a judgment on the filtering pressure trigger/switching under different time constants for pressure filtering, and a change of the trigger threshold in the case of an exceptional gas leakage mutation.

Experiments show that, with the present invention, the human-machine synchrony of the traditional ICU ventilator operating in the NIV mode is equivalent to that of the dedicated non-invasive ventilator, that is, the gas leakage can be completely compensated within 1 to 2 respiration cycles after a gas leakage variation occurs.

The solution of the present invention will be described in detail below in conjunction with the drawings.

Referring to FIG. 2, which is a schematic diagram showing the operating of the invasive ventilator in the non-invasive ventilation mode. The ventilator mechanically ventilates a patient by a double-limb pipeline and a mask. An inspiratory flow sensor for measuring the inspiratory flow and an expiratory flow sensor for measuring the expiratory flow are provided within the ventilator. The airway pressure Paw of the patient can be measured by a pressure sensor arranged at an end of a Y-shaped joint that is close to the patient as shown in FIG. 2, or measured by an inspiratory pressure sensor and an expiratory pressure sensors arranged in the ventilator.

An expiratory valve is closed during the inhaling of the patient, thus gas outputted by the ventilator flows to the patient through an inspiration branch, and possibly a portion of the gas is exhausted via a joint leakage between the mask and the patient's face. The expiratory valve is opened during the exhaling of the patient, the basic flow outputted by the ventilator and the gas exhaled by the patient flow towards the expiratory valve in the ventilator through an expiratory branch, and still possibly a portion of the exhaled gas is exhausted through the joint leakage between the mask and the patient's face. In any of the inspiratory process and the expiratory process, the flows meet the following equation:

fₐₗᵥ=fᵢ−fₗ−fₑ . . . | Formula 1

In Formula 1, fₐₗᵥ| denotes a pulmonary flow, fᵢ denotes the inspiratory flow, fₑ| denotes the expiratory flow, and fₗ denotes the gas leakage flow.

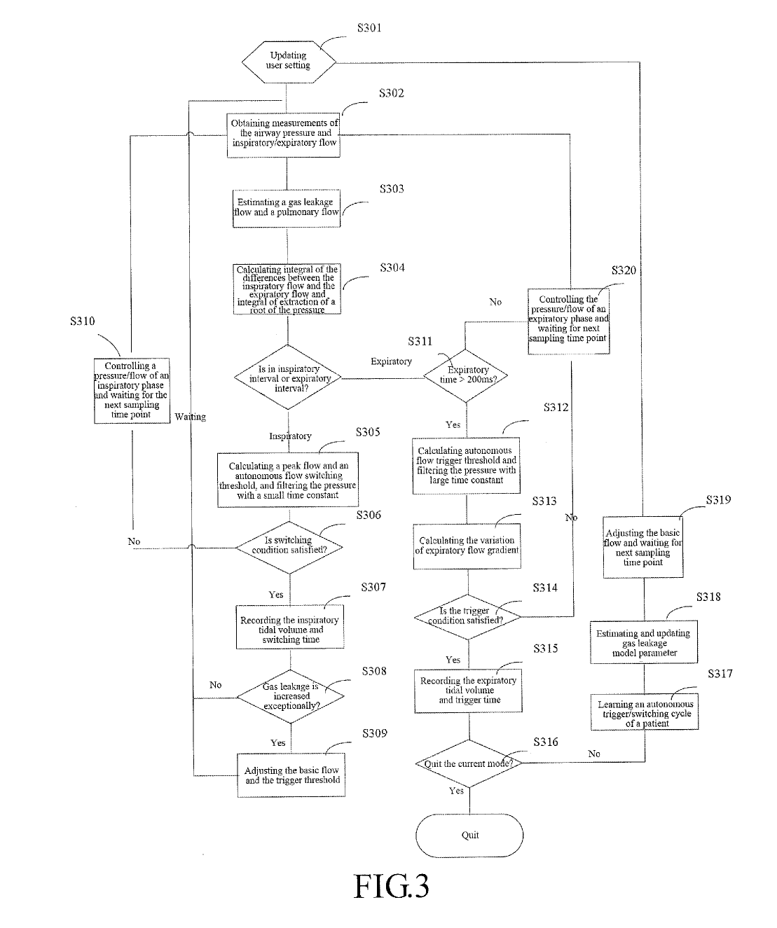

FIG. 3 is a flow chart showing the implementation of a human-machine synchronization method of an invasive ventilator operating in the non-invasive ventilation mode according to the embodiment of the present invention.

A user selects the NIV mode and sets mode parameters corresponding to the NIV mode, such as the inspiratory pressure, the positive end-expiratory pressure, the pressure rising time, the maximum inspiratory time and the trigger/switching threshold (step S301), thus a circulation process is started.

At each sampling time point in any of the inspiratory interval and the expiratory interval, measured values such as the flow and pressure are obtained (S302); then the gas leakage flow and the pulmonary flow are estimated according to parameters of the gas leakage model updated at the end of the last expiration (S303); then the integral of flow differences between the inspiration and the expiration and the integral of extraction of a root of the pressure required by the estimation of the latest parameters of the gas leakage model are calculated (S304); and then it is judged whether the sampling time point is within the inspiratory interval or within the expiratory interval according to the inspiratory/expiratory action of the patient.

If the sampling time point is within the inspiratory interval, the peak flow and the autonomous switching threshold are calculated, and the pressure signal is filtered for a small time constant (S305); and then it is judged whether to transform from the inspiration to the expiration (S306). If the condition for the switching is satisfied, the switching to the expiration is performed, and the inspiratory tidal volume and switching time of the current respiration are recorded (S307), and then it is determined whether an exception of the gas leakage increase occurs (S308). If the exception occurs, then processing corresponding to the exceptional gas leakage mutation is performed (S309), and then the next sampling time point is awaited. If the switching condition is not satisfied, the inspiratory pressure/flow control is performed (S310).

If the sampling time point is within the expiratory interval and the expiratory time has exceeded a preset value which is, for example, 200 ms (S311), the autonomous flow trigger threshold is calculated, and the pressure filtering based on a large time constant is performed (S312); the variation of the expiratory flow gradient is calculated (S313); and then it is judged whether to transform from the expiration to the inspiration (S314). If the condition for the trigger is satisfied, the triggering of the inspiration is performed, and the expiratory tidal volume and trigger time of the current respiration are recorded (S315), then it is judged whether to quit the current mode (S316), and if not, the autonomous switching/trigger cycle of the patient is updated by using the learning mechanism proposed by the present invention (which will be described in detailed below) according to the switching/trigger time recorded at the current and the preceding respiration (S317), then the parameters of the gas leakage model are estimated (S318) and the next expiratory basic flow is updated (S319); if the condition for the trigger is not satisfied, the expiratory pressure/flow control is performed (S320).

Some main technologies of the present invention solution will be described in detail below.

1. Gas Leakage Estimation and Gas Leakage Compensation

Here, assume the gas leakage flow of the mask accord with the following gas leakage estimation model:

fₗ=kₗ·Paw^{0.5} Formula 2

In Formula 2, fₗ| denotes a gas leakage flow, kₗ| denotes a gas leakage model parameter, and P_{aw}| denotes the airway pressure.

The air leakage estimation is to estimate a gas leakage flow (which cannot be measured directly) according to the inspiratory flow, the expiratory flow and the airway pressure that can be measured directly.

According to Formulas 1 and 2, assume the volume of gas inhaled by the patient is approximately equal to the volume of gas exhaled during each respiration, obtaining an approximate equation as follows:

$${V}_{leak} = {\sum }_{i = j}^{j - N + 1} {\int }_{{T}_{i}}^{{T}_{i + 1}} {f}_{i} \cdot d t \approx {\sum }_{i = j}^{j - N + 1} {\int }_{{T}_{i}}^{{T}_{i + 1}} ( {f}_{i} - {f}_{e} ) \cdot d t \approx {k}_{l} \cdot {\sum }_{i = j}^{j - N + 1} {\int }_{{T}_{i}}^{{T}_{i + 1}} {P}_{aw}^{0.5} \cdot d t \vert \qquad Formula 3$$

In Formula 3, Vₗₑₐₖ| denotes the total gas leakage flows in N respirations, j denotes an index of a respiration, Tᵢ denotes a starting time point of the inspiration, and Tᵢ₊₁| denotes a starting time point of the next inspiration. The following estimation formula for parameters of the gas leakage model can be obtained according to Formula 3:

$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} \qquad Formula 4$$

The choosing of the number of respirations N in the above formula is very important. If the gas leakage is relatively stable, N is selectively of a large value, and if the gas leakage is increased or decreased uncommonly, N is selectively of a small value, a preferred example is as follows:

$$\{ \begin{matrix}if & ( \begin{matrix}\vert {\int }_{{T}_{j}}^{{T}_{j + 1}} 1000 \cdot ( {f}_{i} - {f}_{e} - {f}_{l} ) / 60 \cdot d t \vert < \\ 100 ML\end{matrix} ) and ( \begin{matrix}\vert {max}_{j} {f}_{i} - {max}_{j - 1} {f}_{i} \vert < \\ 10 L / min\end{matrix} ) N = 3 \\ else & N = 1\end{matrix} \vert \qquad Formula 5$$

In Formula 5, j| still denotes an index of a respiration, the unit of the flow is L/min (i.e. Liter/minute), the unit for the volume is mL (i.e. milliliter), and the unit of the integrated time is second.

At the end of each expiration, the current parameter kₗ of the gas leakage model can be estimated by Formula 4, and then in the next respiration, the gas leakage flow at each time point can be estimated according to Formula 2. Further, the pulmonary flow fₐₗᵥ can be estimated according to Formula 1. According to the estimated parameters of the gas leakage model, the basic flow of the next expiration can be updated by Formula 6 below:

baseflow(j+1)=baseflowₛₑₜ+kₗ(j)·PEEPₛₑₜ^{0.5} Formula 6

In Formula 6, j still denotes an index of a respiration, PEEPₛₑₜ denotes the set positive end-expiratory pressure, and baseflowₛₑₜ| denotes a set or default basic flow.

2. Learning of Autonomously Trigger/Switching Cycle of a Patient

A patient experiencing the invasive ventilation generally has a strong autonomous respiration ability, therefore, the patient's autonomous respiratory frequency (that is, an autonomous respiratory cycle) is substantially stable in a long period. If the autonomous respiratory cycle of the patient may be acquired, the most sensitive trigger/switching threshold can be set in a period when the patient most likely inhales (or exhales), but a relatively insensitive trigger/switching threshold is set in the other periods. In this way, the trigger/switching power of the patient can be saved maximally while reducing the incorrect triggers/switching.

The autonomous respiratory cycle of the patient may be particularly obtained as follows. At the end of each respiration (that is, the end of each expiration), the switching time and the trigger time of this respiration are recorded; and if the switching time and the trigger time of the current respiration respectively differentiate from the switching time and trigger time of the preceding respiration by less than the predefined value (that is, if the switching/trigger time of the current respiration is very similar to the switching/trigger time of the preceding respiration), the autonomous respiratory frequency of the patient is stable, and the learning of the autonomous trigger/switching cycle of the patient is started; if the switching/trigger time of the current respiration is significantly different from the switching/trigger time of the preceding respiration, the autonomous respiratory frequency of the patient is unstable or the patient does not have the ability of autonomous respiration, thus the learning of the autonomous trigger/switching cycle is not started. Once the process of the learning is started, the subsequent respirations are iterated, until the user changes the mode configuring parameter or until a respiration that is not triggered by the patient.

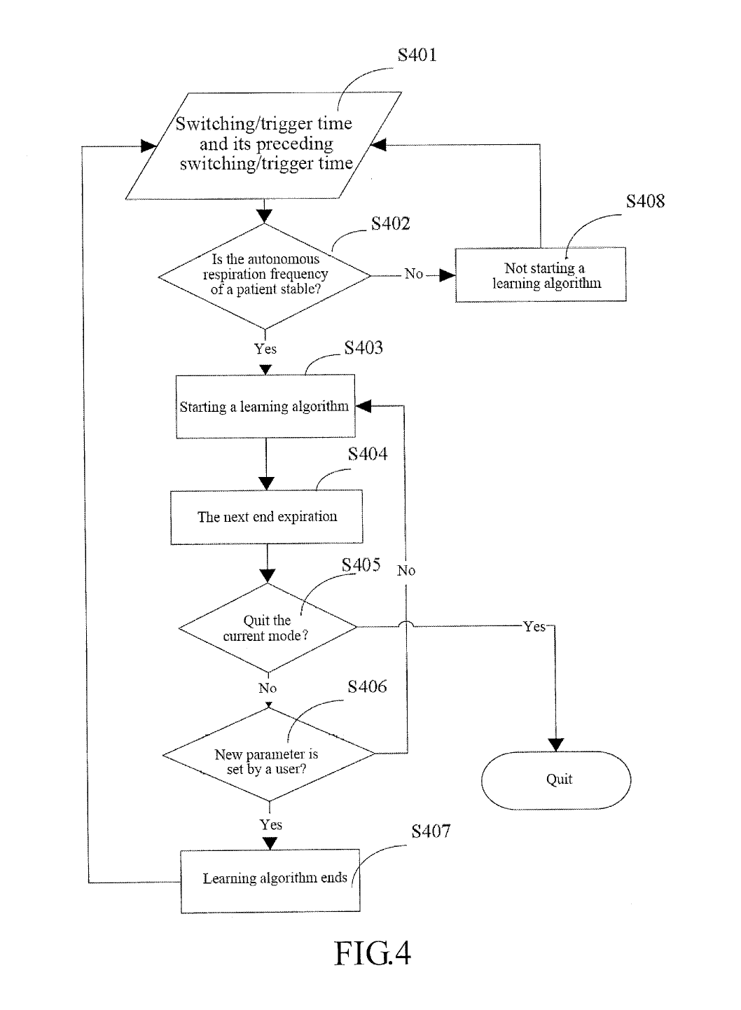

Referring to FIG. 4 which shows a flow chart of learning the autonomous respiratory cycle. Firstly, switching/trigger time at the current respiration and switching/trigger time at the preceding respiration are obtained (S401), then it is judged whether the autonomous respiratory frequency is stable (S402), and if so, the learning of the autonomous trigger/switching cycle of the patient is started (S403), then the next end expiration is awaited (S404), and it is judged whether to quit the current mode (S405); and if so, the current mode is quit, but if the current mode is not quit and the parameters are re-configured by the user (S406), the learning is ended (S407) and the step S401 is performed again; if the parameters are not re-configured by the user, the step S403 is performed again to proceed with the cycle learning; and if the autonomous respiratory frequency of the patient is unstable, the learning will not be started (S408) and then the step S401 is performed again.

The autonomy trigger/switching cycle of the patient is learned iteratively by means of digital filtering, and the following formula may be used:

learn_trig_timeⱼ=α·learn_trig_timeᵢ₋₁+(1−α)·trig_timeⱼ

learn_cyc_timeⱼ=β·learn_cyc_timeᵢ₋₁(1−β)·cyc_time Formula 7

In Formula 7, j denotes an index of a respiration, learn_trig_time denotes the learned autonomous triggering cycle of the patient, learn_cyc_time denotes the learned autonomous switching cycle of the patient, trig_time denotes trigger time at the current respiration, and cyc_time denotes switching time at the current respiration, here, inspiration starting time at a certain respiration is used as a reference for all these four variable. α, β in Formula 7 denotes a constant between 0 and 1.

3. Dynamic Adjustment of Autonomous Trigger/Switching Threshold

For the purpose of saving the trigger/switching power of the patient maximally while reducing incorrect triggers/switching, the autonomous trigger/switching threshold is designed in such a manner that: at the autonomous triggering/switch time point obtained by the learning, the threshold has a value set by the user; and at the other time points, the threshold is designed to be of an insensitive value. The particular calculation formula is as follows:

$$S\_C \_TH = set\_cyc \_TH \cdot exp ( ( t - learn\_cyc \_time ) / 100 ) \\ S\_T \_TH ( t ) = \{ \begin{matrix}20 & t \leq cyc\_time + 300 \\ 20 - ( \begin{matrix}20 - \\ set\_trig - \\ TH\end{matrix} ) \cdot exp ( ( \begin{matrix}t - \\ learn\_trig \_time + \\ 100\end{matrix} ) / 50 ) & cyc\_time + 300 < t \leq learn\_trig \_time - 100 \\ set\_trig \_TH & else\end{matrix} \qquad Formula 8$$

In Formula 8, t denotes a certain time point during the respiration, with the starting time of the inspiration within the respiration being used as a reference, S_C_TH denotes the autonomous switching threshold, S_T_TH denotes the autonomous trigger threshold, set_cyc_TH denotes a switching threshold (the percentage of the peak flow) set by the user, and set_trig_TH denotes the trigger threshold (i.e. a flow, or a flow volume) set by the user. In Formula 8, the unit of the flow is Liter/minute, and the unit of the time is milliseconds.

The graph of FIG. 5 shows the autonomous trigger/switching threshold versus respiratory time. It can be seen from Formula 8 and FIG. 5, the autonomous trigger threshold decreases exponentially along with the time, and the autonomous switching threshold increases exponentially along with the time. However, it should be known that the curve of the threshold versus time may be varied and is not limited to an exponential function, according to the concept of the present invention.

4. Calculation of the Variation of the Expiratory Flow Gradient

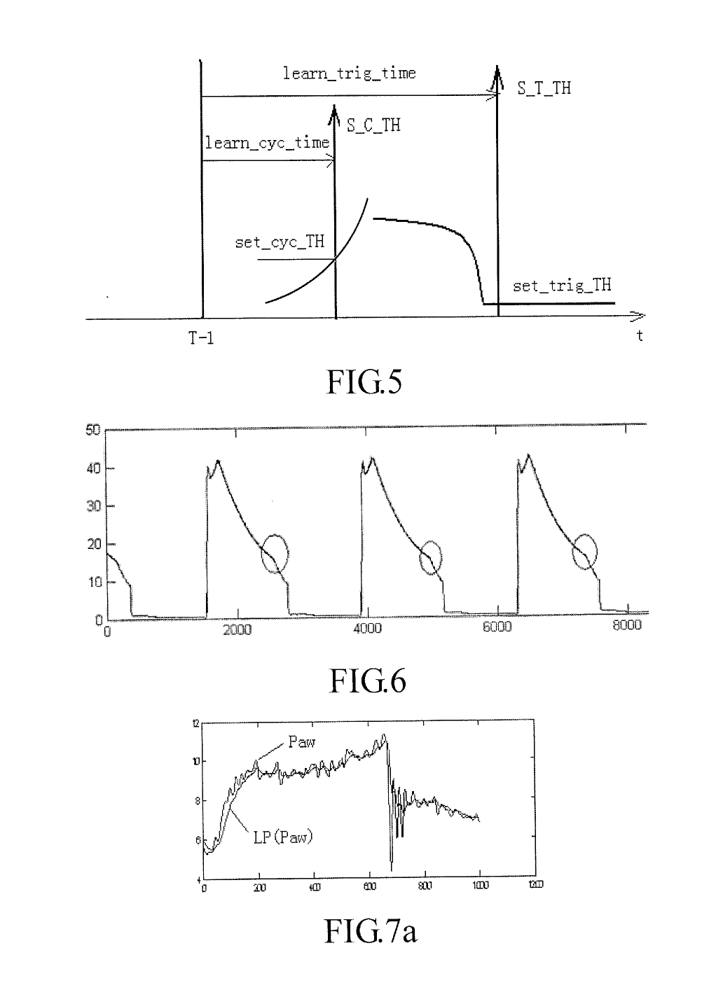

The trigger mechanism based on the variation of the expiratory flow gradient is based on such a fact that: an obvious inflection point is present on the waveform of the expiratory flow when the patient exerts himself to inhale, as shown in FIG. 6 which shows the waveform of the expiratory flow measured on an Active Servo Lung ASL15000 experiencing ventilation in the presence of gas leakage. Due to the little affection on this feature information (i.e. the inflection point) by the gas leakage, incorrect triggers caused by the gas leakage can be reduced by the trigger judgment based on the feature information.

The expiratory flow gradient is equivalent to the difference of the expiratory flow, that is, fe(T−ΔT)−fe(T), or the variation of the expiratory flow during a period of time ΔT. Here, ΔT has a value of 50 ms, for example. The variation of the expiratory flow gradient is equivalent to a second-order difference of the expiratory flow, that is, 2*fe(T−ΔT)−fe(T−2ΔT)−fe(T)|. It should be known from the definition of the gradient and FIG. 5 that the expiratory flow gradient is always decreasing in the expiratory phase, but increases at the inflection point. Therefore, the trigger criterion based on the variation of the expiratory flow gradient is defined as (2*fe(T−ΔT)−fe(T−2ΔT)−fe(T))>2|, here the unit of the flow is Liter/minutes.

5. Pressure Filtering with Different Time Constants During the Inspiratory Phase/Expiratory Phase

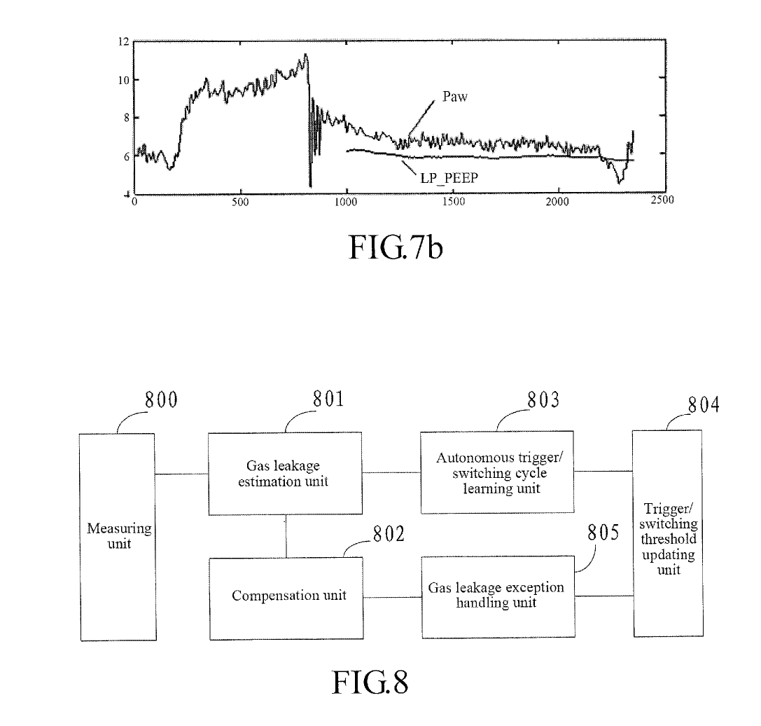

The pressure signal is less influenced by the gas leakage relative to the flow, but the pressure varies significantly when the patient exerts oneself to inhale or exhale. Therefore, the excess of the pressure signal over a certain threshold can be used as an alternate criterion for judging the trigger/switching. In order to reduce the influence of the interference signal mixed in the pressure signal on the correct switching judgment, the pressure in the expiratory phase is subjected to a low-pass filtering treatment in the present invention. However, in order not to affect the tendency of the pressure variation, the time constant for the filtering is relatively small, as shown in FIG. 7a. The purpose of the pressure filtering in the expiratory phase is to monitor the actual PEEP of the expiratory phase. Since the PEEP is of a constant value, the time constant of the pressure filter for the expiratory phase is relatively large, as shown in FIG. 7b. As for the filtering pressure as shown in FIG. 7b, the influence of the pressure drop caused by the expiratory flow passing through the expiratory valve on the PEEP measurement also is considered by the specific formula as follows:

LP_PEEPᵢ=0.99·LP_PEEPᵢ₋₁+0.01·(Paw−Rₑ·fₑ) Formula 9

In Formula 9, i denotes a sampling time point, LP_PEEP denotes a value of the measured Positive End-Expiratory Pressure (PEEP), P_{aw}| denotes a pressure value, Rₑ| denotes an air resistance at the expiratory valve, and fₑ| denotes the expiratory flow. The sampling time interval of the applicable digital filter is 1 ms, and the time constant is 0.1 s.

6. Switching Judgment

The switching refers to the transition from the inspiration to the expiration, and may be based on a plurality of judgment conditions of information features, including that: the ratio of the inspiratory flow to the peak flow is lower than the autonomous switching threshold, or the inspiratory filtering pressure is higher than a set pressure threshold, or the actual inspiratory time exceeds a set maximum inspiratory time. The judgment conditions are not prioritized, and the transition to the expiration is made provided that any one of the conditions is satisfied.

7. Trigger Judgment

The trigger refers to the transition from the expiration to the inspiration. Like the switching, the trigger is also based on a plurality of judgment conditions of information features, including that: an estimated pulmonary flow is higher than the autonomous trigger threshold, or the difference between the expiratory pressure and the monitored PEEP exceeds a set pressure threshold, or the trigger criterion of the variation of the expiratory gradient is satisfied, or the expiration time is longer than a preset value thus starting the backup ventilation. The judgment conditions are not prioritized, and the transition to the inspiration is made provided that any one of the conditions is satisfied.

8. Handling of an Exception of Gas Leakage Increase

In the above-described solution of the gas leakage estimation and gas leakage compensation, the gas leakage parameter estimation and gas leakage compensation are performed after each trigger (i.e. at the beginning of each inspiration), therefore, an incorrect trigger of the next inspiration may be caused if an exception of the gas leakage increase during a certain inspiratory phase is not handled timely. In the present invention, whether the exception of the gas leakage increase occurs is judged according to the current inspiratory tidal volume and the preceding inspiratory tidal volume, and then the basic flow and the trigger threshold during the expiratory phase are adjusted accordingly by a specific modification value as shown in Formula 10 below:

$$leak\_change = \frac{{VI}_{j} - {VI}_{j - 1}}{cyc\_time} \cdot {( \frac{PEEP}{Pc} )}^{0.5} \cdot 60 if \\ ( ( {VI}_{j} - {VI}_{j - 1} ) > 100 ) \vert \qquad Formula 10$$

In Formula 10, j denotes an index of a respiration, leak change denotes the modification value by which the basic flow and the trigger threshold during the expiratory phase are modified in the case of the exception of the gas leakage increase, VI denotes the inspiratory tidal volume, cyc_time denotes the time for switching from the current inspiration to the expiration, Pₑ| denotes the set pressure during the inspiratory phase, and PEEP denotes the Positive End-Expiratory airway Pressure. In Formula 10, the unit of the tidal volume is mL, the unit of the time is milliseconds, and the unit of the flow is L/min.

The present invention also provides a device for human-machine synchronization of an invasive ventilator operating in the non-invasive ventilation mode, which corresponds to the method above. It should be understood by those skilled in the art that the device, which controls the ventilator, may be integrated in or separated from the ventilator, and can be implemented by hardware, software or a combination of the hardware and the software.

FIG. 8 is a schematic diagram showing the structure of the above device. The device includes:

a measuring unit 800, which is used for measuring an airway pressure, an inspiratory flow and a respiratory flow;

a gas leakage estimation unit 801, which is used for calculating a gas leakage flow based on the airway pressure, the inspiratory flow and the expiratory flow measured by the measuring unit 800, according to the pre-established gas leakage estimation model; and

a compensation unit 802, which is used for compensating a basic flow according to the gas leakage flow.

The specific principle and calculation formulas of the gas leakage compensation are the same as those described with reference to the method embodiment, specifically the description of Formulas 1 to 5.

Preferably, the device further includes:

an autonomous trigger/switching cycle learning unit 803, which is used for recording the switching time and the trigger time of the current respiration at the end of expiration, iteratively learning the trigger/switching cycle by means of digital filtering to obtain the autonomy trigger/switching cycle of a patient if the switching time and the trigger time of the current respiration respectively differentiate from the switching time and the trigger time of the preceding respiration by less than a predefined value, and updating parameters of the gas leakage estimation model according to the latest autonomy trigger/switching cycle of the patient.

Further, the device further includes:

a trigger/switching threshold updating unit 804, which is used for setting the trigger/switching threshold to be a threshold with a high sensitivity at the autonomy trigger/switching time point and to be a threshold with a low sensitivity at other time points according to the autonomous trigger/switching cycle of the patient obtained by the autonomous trigger/switching cycle learning unit.

In addition, the device may further include:

a gas leakage exception handling unit 805, which is used for judging whether an exception of the gas leakage increase is present according to the current inspiratory tidal volume and the preceding inspiratory tidal volume at the end of the inspiration, and if so, instructing the compensation unit 802 and the trigger/switching threshold updating unit 804 to adjust the basic flow and the trigger threshold of the expiratory phase.

As can be seen, in the present invention, the gas leakage flow is estimated by the gas leakage estimation model for the purpose of the gas leakage compensation, the parameters of the gas leakage estimation model are updated in each respiration cycle, and the number of respirations N needed by the parameter estimation are adaptively selected according to the gas leakage variation, thereby achieving the relatively accurate gas leakage estimation by using the estimated parameters. Further, the basic flow and the trigger threshold are adjusted when the gas leakage is exceptionally increased, thereby assuring relatively accurate estimation of the gas leakage flow and the pulmonary flow even in the case of a large gas leakage or a gas leakage exception.

Further, the present invention provides the mechanism of learning the autonomous trigger/switching cycle of the patient, so that the autonomous trigger/switching cycle of the patient can be used as a basis of the trigger/switching threshold. The autonomous trigger/switching threshold is not fixed, but varied with time, thereby reducing the trigger/switching power of the patient maximally while preventing an incorrect trigger/switching.

In addition, the variation of the expiratory flow gradient is used as the criterion of the trigger judgment in the present invention, to avoid the affection of the gas leakage on the trigger, thus further improving the performance of the man-machine synchrony. Meanwhile, the difference between the PEEP acquired by the filtering algorithm and the actual airway pressure is taken as another criterion of the trigger judgment, to avoid the affection of the gas leakage on the trigger, thus improving the human-machine trigger synchrony.

The preferred embodiments and the technology principle have been described above. Any variation and replacement occurs to those skilled in the art without departing from the scope of the present invention should be included in the scope of the invention.

1. A human-machine synchronization method of an invasive ventilator operating in a non-invasive ventilation mode, comprising:
measuring an airway pressure, an inspiratory flow and an expiratory flow;
calculating a gas leakage flow based on the airway pressure, the inspiratory flow and the expiratory flow according to a pre-established gas leakage estimation model; and
compensating a basic flow according to the gas leakage flow.

2. The method of claim 1, further comprising:
recording switching time and trigger time of the current respiration at the end of expiration, and iteratively learning a trigger/switching cycle by means of digital filtering when the switching time and the trigger time of the current respiration respectively differentiate from the switching time and trigger time of the preceding respiration by less than a preset difference, to obtain an autonomy trigger/switching cycle of a patient.

3. The method of claim 2, further comprising:
setting the trigger/switching threshold to be a threshold with a high sensitivity at the autonomy trigger/switching time point and to be a threshold with a low sensitivity at other time points according to the autonomous trigger/switching cycle of the patient.

4. The method of claim 2, comprising: updating parameters of the gas leakage estimation model according to the latest autonomy trigger/switching cycle of the patient.

5. The method of claim 1, further comprising:
judging, at the end of the inspiration, whether gas leakage is exceptionally increased according to the current inspiratory tidal volume and the preceding inspiratory tidal volume, and if so, modifying the basic flow and the trigger threshold of the expiratory phase.

6. The method of claim 2, wherein the criterion of trigger judgment comprises variations of the expiratory flow gradient or an expiratory filtering pressure with different time constants for pressure filtering; and criterion of switching judgment comprises an inspiratory filtering pressure with different time constants for pressure filtering.

7. The method of claim 1, wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model;
kₗ is calculated by the following parameter estimation model of
$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$
wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations, wherein, the value of N is selected by such a criterion that: N takes a larger value when the gas leakage is stable and takes a smaller value when the gas leakage is increased/decreased exceptionally.

8. A device for human-machine synchronization of an invasive ventilator operating in a non-invasive ventilation mode, comprising:
a measuring unit, which is used for measuring an airway pressure, an inspiratory flow and a respiratory flow;
a gas leakage estimation unit, which is used for calculating a gas leakage flow based on the airway pressure, the inspiratory flow and the expiratory flow measured by the measuring unit according to a pre-established gas leakage estimation model; and
a compensation unit, which is used for compensating a basic flow according to the gas leakage flow.

9. The device of claim 8, further comprising:
an autonomous trigger/switching cycle learning unit, which is used for recording switching time and trigger time of the current respiration at the end of expiration, iteratively learning a trigger/switching cycle by means of digital filtering when the switching time and the trigger time of the current respiration respectively differentiate from the switching time and the trigger time of the preceding respiration by less than a preset difference to obtain an autonomy trigger/switching cycle of a patient, and updating parameters of the gas leakage estimation model according to the latest autonomy trigger/switching cycle of the patient.

10. The device of claim 9, further comprising:
a trigger/switching threshold updating unit, which is used for setting the trigger/switching threshold to be a threshold with a high sensitivity at the autonomy trigger/switching time point and to be a threshold with a low sensitivity at other time points according to the autonomous trigger/switching cycle of the patient obtained by the autonomous trigger/switching cycle learning unit, and/or
a gas leakage exception handling unit, which is used for judging, at the end of the inspiration, whether gas leakage is exceptionally increased according to the current inspiratory tidal volume and the preceding inspiratory tidal volume, and if so, instructing the compensation unit and the trigger/switching threshold updating unit to adjust the basic flow and the trigger threshold of the expiratory phase.

11. The method of claim 3, wherein the criterion of trigger judgment comprises variations of the expiratory flow gradient or an expiratory filtering pressure with different time constants for pressure filtering; and criterion of switching judgment comprises an inspiratory filtering pressure with different time constants for pressure filtering.

12. The method of claim 4, wherein the criterion of trigger judgment comprises variations of the expiratory flow gradient or an expiratory filtering pressure with different time constants for pressure filtering; and criterion of switching judgment comprises an inspiratory filtering pressure with different time constants for pressure filtering.

13. The method of claim 5, wherein the criterion of trigger judgment comprises variations of the expiratory flow gradient or an expiratory filtering pressure with different time constants for pressure filtering; and criterion of switching judgment comprises an inspiratory filtering pressure with different time constants for pressure filtering.

14. The method of claim 2, wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model;
kₗ is calculated by the following parameter estimation model of
$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$
wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations, wherein, the value of N is selected by such a criterion that: N takes a larger value when the gas leakage is stable and takes a smaller value when the gas leakage is increased/decreased exceptionally.

15. The method of claim 3, wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model;
kₗ is calculated by the following parameter estimation model of
$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$
wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations, wherein, the value of N is selected by such a criterion that: N takes a larger value when the gas leakage is stable and takes a smaller value when the gas leakage is increased/decreased exceptionally.

16. The method of claim 4, wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model;
kₗ is calculated by the following parameter estimation model of
$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$
wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations, wherein, the value of N is selected by such a criterion that: N takes a larger value when the gas leakage is stable and takes a smaller value when the gas leakage is increased/decreased exceptionally.

17. The method of claim 5, wherein the gas leakage estimation model is fₗ=kₗ·Paw^{0.5}|; wherein, fₗ denotes the gas leakage flow, P_{aw}| denotes the airway pressure, and kₗ· denotes a parameter of a gas leakage model;
kₗ is calculated by the following parameter estimation model of
$${k}_{l} = \frac{{\sum }_{i = j}^{j - N + 1} {\int }_{{T}_{i}}^{{T}_{i + 1}} {P}_{aw}^{0.5} \cdot d t}{{\sum }_{i = j}^{j - N + 1} {\int }_{{T}_{i}}^{{T}_{i + 1}} ( {f}_{i} - {f}_{e} ) \cdot d t} ;$$
wherein, j| denotes an index of a respiration, Tᵢ| denotes a beginning time point of the inspiration, Tᵢ₊₁| denotes a beginning time point of the next inspiration, fᵢ denotes the inspiratory flow, fₑ| denotes the expiratory flow, and N denotes the number of respirations, wherein, the value of N is selected by such a criterion that: N takes a larger value when the gas leakage is stable and takes a smaller value when the gas leakage is increased/decreased exceptionally.
